(19) United States
(12) Patent Application Publication
(10) Pub. No.: US 2023/0072588 A1
(43) Pub. Date: Mar. 9, 2023

(54) SYSTEMS AND METHODS FOR WET AIR OXIDATION REGENERATION OF CATALYSTS

(71) Applicants: VIRENT, INC., Madison, WI (US); JOHNSON MATTHEY DAVY TECHNOLOGIES LIMITED, London (GB)

(72) Inventors: Paul G. Blommel, Madison, WI (US); Colin Anson, Madison, WI (US); Matt van Straten, Madison, WI (US); Edgar Steenwinkel, Madison, WI (US); Chris Holland, Madison, WI (US); Rauf Gearing, LONDON (GB); Chris Ferguson, LONDON (GB); Rob Wild, LONDON (GB); Ian Campbell, LONDON (GB)

(21) Appl. No.: 17/891,093

(22) Filed: Aug. 18, 2022

Related U.S. Application Data
(60) Provisional application No. 63/235,037, filed on Aug. 19, 2021.

(51) Int. Cl. B01J 38/70; B01J 23/46; B01J 23/96; B01J 21/18; C13K 1/02

(52) U.S. Cl. CPC B01J 38/70; B01J 23/462; B01J 23/96; B01J 21/18; C13K 1/02

(57) ABSTRACT

The present disclosure provides methods for producing a regenerated hydrogenation catalyst from a fouled hydrogenation catalyst having a total surface area and at least one associated impurity. The method can include maintaining contact between the fouled hydrogenation catalyst and a flushing medium that comprises water, oxygen, and an inert or diluent gas at a regeneration temperature and a regeneration pressure sufficient to remove at least a portion of the at least one impurity from the hydrogenation catalyst to produce the regenerated hydrogenation catalyst, where the regenerated hydrogenation catalyst is characterized as retaining at least 70% of the activity of the hydrogenation catalyst.

### CROSS-REFERENCE TO RELATED APPLICATIONS

This application claims priority to U.S. Provisional Application No. 63/235,037, filed Aug. 19, 2021, the content of which is hereby incorporated by reference in its entirety.

### BACKGROUND

Increasing cost of fossil fuel and environmental concerns have stimulated worldwide interest in developing alternatives to petroleum-based fuels, chemicals, and other products. Biomass is one category of possible renewable alternatives to such fuels and chemicals.

A key challenge for promoting and sustaining the use of biomass in the industrial sector is the need to develop efficient and environmentally benign technologies for converting biomass to useful products. Biomass conversion technologies unfortunately tend to carry additional costs, which make it difficult to compete with products produced through the use of traditional resources, such as fossil fuels. Such costs often include capital expenditures on equipment and processing systems capable of sustaining extreme temperatures and high pressures, and the necessary operating costs of heating fuels and reaction products, such as fermentation organisms, enzymatic materials, catalysts and other reaction chemicals.

Bioreforming processes address these issues and provide liquid fuels and chemicals derived from the cellulose, hemicellulose and lignin found in plant cell walls. For instance, cellulose and hemicellulose can be used as feedstock for various bioreforming processes, including aqueous phase reforming (APR) and hydrodeoxygenation (HDO)—catalytic reforming processes that, when integrated with hydrogenation, can convert cellulose and hemicellulose into hydrogen and hydrocarbons, including liquid fuels and other chemical products. APR and HDO methods and techniques are described in U.S. Pat. Nos. 6,699,457; 6,964,757; 6,964,758; and 7,618,612 (all to Cortright et al., entitled “Low-Temperature Hydrogen Production from Oxygenated Hydrocarbons”); U.S. Pat. No. 6,953,873 (to Cortright et al., entitled “Low-Temperature Hydrocarbon Production from Oxygenated Hydrocarbons”); and U.S. Pat. Nos. 7,767,867 and 7,989,664 and U.S. Application No. 2011/0306804 (all to Cortright, entitled “Methods and Systems for Generating Polyols”). Various APR and HDO methods and techniques are described in U.S. Pat. Nos. 8,053,615; 8,017,818 and 7,977,517 and U.S. patent application Ser. Nos. 13/163,439; 13/171,715; 13/163,142 and 13/157,247 (all to Cortright and Blommel, entitled “Synthesis of Liquid Fuels and Chemicals from Oxygenated Hydrocarbons”); U.S. Patent Application No. 2009/0211942 (to Cortright, and entitled “Catalysts and Methods for Reforming Oxygenated Compounds”); U.S. Patent Application No. 2010/0076233 (to Cortright et al., entitled “Synthesis of Liquid Fuels from Biomass”); International Patent Application No. PCT/US2008/056330 (to Cortright and Blommel, entitled “Synthesis of Liquid Fuels and Chemicals from Oxygenated Hydrocarbons”); and commonly owned co-pending International Patent Application No. PCT/US2006/048030 (to Cortright et al., entitled “Catalyst and Methods for Reforming Oxygenated Compounds”), all of which are incorporated herein by reference.

In certain applications, it may be beneficial for biomass feedstock to be hydrogenated to increase the thermal stability of the biomass feedstock prior to use as a feed for APR and/or HDO. At temperatures compatible with APR and/or HDO, sugars are susceptible to thermal degradation, which leads to byproduct formation, catalyst fouling, and, ultimately, shortened time between catalyst regenerations. This problem is avoided by reacting sugars with hydrogen to form polyols or sugar alcohols that are more thermally stable.

Biomass feedstock includes impurities, such as sulfur-containing moieties, that poison hydrogenation catalysts over time. Poisoning of the catalyst leads to lower conversion and yield of polyol and sugar alcohol products. As a result, most industrial applications involve a batch or semi-continuous process that involves changing the spent catalyst with fresh catalyst or regenerating the existing catalyst to improve conversion. Changing the hydrogenation catalyst frequently is time consuming, expensive, and can lead to production downtime.

Current methods for regenerating hydrogenation catalysts include using multiple hydrogen peroxide washes to remove impurities from the spent hydrogenation catalyst. However, hydrogen peroxide damages the physical strength of the catalyst over time, reducing both total surface area and, ultimately, catalytic activity.

### SUMMARY OF THE INVENTION

Described herein are reactor systems and methods for regenerating hydrogenation catalysts for use in hydrogenating feedstock solutions, such as water-soluble sugars derived from biomass and/or unsaturated hydrocarbon streams. The provided reactor systems and methods offer unique features and advantages over existing regeneration techniques. In some embodiments, the provided reactor systems and methods for regenerating hydrogenation catalysts offer mild reaction conditions that can effectively remove impurities to restore hydrogenation catalytic activity, while additionally maintaining the catalyst's structural integrity (e.g., surface area, pore volume). Maintaining the catalyst's structural integrity and/or catalytic activity for extended periods of time improves operation economics by reducing the number of times the catalyst needs to be replaced over time, and by reducing regeneration frequency. This is an improvement over current techniques to regenerate catalytic activity, such as hydrogen peroxide based methods, which have a tendency to degrade the catalyst's surface area and pore structure over time. Further, hydrogen peroxide poses storage challenges on a commercial scale. The regenerative oxidants provided herein are cheaper than hydrogen peroxide, and can be stored at commercial scale using existing technology.

In one embodiment, the present disclosure provides a method for producing a regenerated hydrogenation catalyst from a fouled hydrogenation catalyst having a total surface area, an activity, and at least one associated impurity. The method includes maintaining contact between the fouled hydrogenation catalyst and a flushing medium that comprises water and oxygen at a regeneration temperature and a regeneration pressure sufficient to remove at least a portion of the at least one impurity from the hydrogenation catalyst to produce the regenerated hydrogenation catalyst, where the regenerated hydrogenation catalyst is characterized as retaining at least 70% of the total surface area of the fouled hydrogenation catalyst or is characterized by retaining at least 70% of the substrate conversion activity of the fouled hydrogenation catalyst.

In another embodiment, the present disclosure provides a method for hydrogenating a biomass stream. The method includes catalytically reacting a feedstock stream comprising water and sugar with hydrogen in the presence of a hydrogenation catalyst for a hydrogenation duration to produce a fouled hydrogenation catalyst. The method further includes replacing the feedstock stream with a flushing medium comprising water and oxygen and maintaining contact between the fouled hydrogenation catalyst and the flushing medium at a regeneration temperature and a regeneration pressure for a regeneration duration to produce a regenerated hydrogenation catalyst, where the regenerated hydrogenation catalyst is characterized as retaining at least 70% of the total surface area of the fouled hydrogenation catalyst or is characterized by retaining at least 70% of the substrate conversion activity of the fouled hydrogenation catalyst.

In one embodiment, the present disclosure provides a method for hydrogenating a biomass stream. The method includes catalytically reacting a feedstock stream comprising water and an oxygenated hydrocarbon (e.g., C₂₊O₁₊) with hydrogen in the presence of a hydrogenation catalyst for a hydrogenation duration to produce a fouled hydrogenation catalyst. The method includes replacing the feedstock stream with a flushing medium comprising water and oxygen, and maintaining contact between the fouled hydrogenation catalyst and the flushing medium at a regeneration temperature and a regeneration pressure for a regeneration duration to produce a regenerated hydrogenation catalyst. The regenerated hydrogenation catalyst is characterized as retaining at least 70% of the conversion of the hydrogenation catalyst for the oxygenated hydrocarbon in the feedstock after contacting the flushing medium to the hydrogenation catalyst for at least 1 hour at the regeneration temperature and the regeneration pressure. For example, the regenerated hydrogenation catalyst is characterized as retaining more than 100% of the conversion of the fouled hydrogenation catalyst for the oxygenated hydrocarbon in the feedstock and retaining at least 70% of the conversion of the hydrogenation catalyst for the oxygenated hydrocarbon in the feedstock after contacting the flushing medium to the hydrogenation catalyst for at least 1 hour at the regeneration temperature and the regeneration pressure.

In another embodiment, the present disclosure provides a method for regenerating a fouled hydrogenation catalyst having at least one associated impurity. The includes maintaining contact between the fouled hydrogenation catalyst and a flushing medium that comprises water, oxygen, and an inert gas at a regeneration temperature and a regeneration pressure sufficient to remove at least a portion of the at least one impurity from the hydrogenation catalyst to produce a regenerated hydrogenation catalyst, wherein a conversion of the regenerated hydrogenation catalyst for an oxygenated hydrocarbon (C₂₊O₁₊) is higher than a conversion of the fouled hydrogenation catalyst for the oxygenated hydrocarbon. For example, the conversion of the regenerated hydrogenation catalyst can be at least 5%, at least 10%, at least 50%, or at least 100% higher than the conversion of the fouled hydrogenation catalyst.

In another embodiment, the present disclosure provides a method for hydrogenating a biomass stream. The method includes catalytically reacting a feedstock stream comprising water and sugar with hydrogen in the presence of a hydrogenation catalyst for a hydrogenation duration to produce a fouled hydrogenation catalyst. The method further includes replacing the feedstock stream with a flushing medium comprising water, oxygen, and an inert gas. The method further includes maintaining contact between the fouled hydrogenation catalyst and the flushing medium at a regeneration temperature and a regeneration pressure for a regeneration duration to produce a regenerated hydrogenation catalyst, wherein a conversion of the regenerated hydrogenation catalyst for an oxygenated hydrocarbon (C₂₊O₁₊) is higher than a conversion of the fouled hydrogenation catalyst for the oxygenated hydrocarbon. For example, the conversion of the regenerated hydrogenation catalyst can be at least 5%, at least 10%, at least 50%, or at least 100% higher than the conversion of the fouled hydrogenation catalyst.

In another embodiment, the present disclosure provides a method for producing a regenerated hydrogenation catalyst from a fouled hydrogenation catalyst, the fouled hydrogenation catalyst having at least one sulfur-containing impurity. The method includes catalytically reacting a feedstock stream having at least one sulfur-containing impurity in the presence of a hydrogenation catalyst for a hydrogenation duration to produce the fouled hydrogenation catalyst. The method further includes replacing the feedstock stream with a flushing medium comprising water and oxygen and maintaining contact between the fouled hydrogenation catalyst and the flushing medium at a regeneration temperature and a regeneration pressure for a regeneration duration to produce a regenerated hydrogenation catalyst. The concentration of the at least one sulfur-containing impurity in the regenerated hydrogenation catalyst is reduced relative to the fouled hydrogenation catalyst. In some embodiments, the regeneration temperature is from 50° C. to 200° C. In some embodiments, the regeneration pressure from 20 psig to 300 psig. In some embodiments, the regeneration temperature is from 50° C. to 200° C. and the regeneration pressure from 20 psig to 300 psig.

In one embodiment, the present disclosure provides a method for producing a regenerated hydrogenation catalyst from a fouled hydrogenation catalyst, the fouled hydrogenation catalyst having at least one carbon-containing impurity. The method includes catalytically reacting a feedstock stream having at least one carbon-containing impurity in the presence of a hydrogenation catalyst for a hydrogenation duration to produce the fouled hydrogenation catalyst. The method includes replacing the feedstock stream with a flushing medium comprising water and oxygen, and maintaining contact between the fouled hydrogenation catalyst and the flushing medium at a regeneration temperature and a regeneration pressure for a regeneration duration to produce a regenerated hydrogenation catalyst. The concentration of the at least one carbon-containing impurity in the regenerated hydrogenation catalyst is reduced relative to the fouled hydrogenation catalyst.

In another embodiment, the present disclosure provides a method for producing a regenerated hydrogenation catalyst from a fouled hydrogenation catalyst, the fouled hydrogenation catalyst having at least one sulfur-containing impurity. The method includes catalytically reacting a feedstock stream having at least one sulfur-containing impurity in the presence of a hydrogenation catalyst to produce the fouled hydrogenation catalyst. The method includes replacing the feedstock stream with a flushing medium characterized in that the flushing medium comprises a liquid phase and a vapor phase, wherein the liquid phase comprises water and the vapor phase comprises oxygen, and maintaining contact between the fouled hydrogenation catalyst and the flushing medium at a regeneration temperature from 50° C. to 200° C., and a regeneration pressure from 20 psig to 300 psig for a regeneration duration to produce a regenerated hydrogenation catalyst. The concentration of the at least one sulfur-containing impurity in the regenerated hydrogenation catalyst is reduced relative to the fouled hydrogenation catalyst.

In the method according to any one of the preceding embodiments, the regenerated hydrogenation may be characterized as retaining at least 70% of the total surface area of the fouled hydrogenation catalyst after contacting the flushing medium to the fouled hydrogenation catalyst for at least 1 hour, or at least 6 hours, or at least 12 hours, or at least 24 hours, or at least 2 days, or at least one week at the regeneration temperature and the regeneration pressure.

In the method according to any one of the preceding embodiments, the regenerated hydrogenation catalyst may be characterized as retaining at least 80%, or at least 90%, or at least 95% of the total surface area of the fouled hydrogenation catalyst after contacting the flushing medium to the fouled hydrogenation catalyst for at least 1 hour at the regeneration temperature and the regeneration pressure.

In the method according to any one of the preceding embodiments, the regenerated hydrogenation catalyst may be characterized as exhibiting at least a 5% reduction in a specified impurity (e.g., sulfur-containing impurity or carbon-containing impurity) relative to the fouled hydrogenation catalyst.

In the method according to any one of the preceding embodiments, the regenerated hydrogenation catalyst may be characterized as exhibiting at least a 5% reduction in the impurity relative to the fouled hydrogenation catalyst after contacting the flushing medium to the fouled hydrogenation catalyst for at least 1 hour, or at least 6 hours, or at least 12 hours, or at least 24 hours, or at least 2 days, or at least one week at the regeneration temperature and the regeneration pressure.

In the method according to any one of the preceding embodiments, the regenerated hydrogenation catalyst may be characterized as exhibiting at least a 10% reduction, or at least a 15% reduction, or at least a 20% reduction, or at least a 25% reduction in the impurity relative to the fouled hydrogenation catalyst after contacting the flushing medium after contacting the flushing medium to the hydrogenation catalyst at the regeneration temperature and the regeneration pressure.

In the method according to any one of the preceding embodiments, the regeneration pressure may range from 20 psig to 300 psig and/or the regeneration temperature may range from 50° C. to 200° C.

In the method according to any one of the preceding embodiments, the flushing medium may comprise a liquid phase and a vapor phase. The vapor phase may comprise an oxygen content from 0.1% to 40% (v/v), e.g., an oxygen content of at least 1% (v/v), or at least 5% (v/v), or at least 10% (v/v), or at least 15% (v/v), or at least 20% (v/v), or at least 25% (v/v).

In the method according to any one of the preceding embodiments, the inert gas (e.g., nitrogen) may be present in the vapor phase in an amount from 60% (v/v) to 99.5% (v/v). The method according to any one of the preceding embodiments may include a vapor phase that comprises air.

In the method according to any one of the preceding embodiments, the flushing medium may include an oxygen to catalyst flux ratio (O₂/cat/hr) from 0.1*10⁻³ to 100*10⁻³ (mols/w/hr), or from 0.1*10⁻³ to 10*10⁻³ (mols/w/hr).

In the method according to any one of the preceding embodiments, the flushing medium may have a water to catalyst flux ratio (H₂O/cat/hr) from 1 to 100 (w/w/hr).

In the method according to any one of the preceding embodiments, the flushing medium may be free of hydrogen peroxide.

In the method according to any one of the preceding embodiments, the hydrogenation catalyst includes a support and an active metal. The hydrogenation catalyst may have at least one of the following properties (i) a total surface area of at least 500 m²/g; (ii) a micropore surface area of at least 400 m²/g; and (iii) a mesopore surface area of at least 30 m²/g.

In the method according to any one of the preceding embodiments, the regenerated hydrogenation catalyst may have at least one of the following properties (i) the regenerated hydrogenation catalyst is characterized by retaining at least 70% of the micropore surface area of the fouled hydrogenation catalyst after contacting the flushing medium for at least 1 hour at the regeneration temperature and the regeneration pressure; and (ii) the regenerated hydrogenation catalyst is characterized by retaining at least 70% of the mesopore surface area of the fouled hydrogenation catalyst after contacting the flushing medium for at least 1 hour at the regeneration temperature and the regeneration pressure.

In the method according to any one of the preceding embodiments, the hydrogenation catalyst may be ruthenium on carbon (Ru/C).

### BRIEF DESCRIPTION OF DRAWINGS

FIG. 1 is an example reactor system in accordance with some embodiments of the present disclosure.

FIG. 2 illustrates wet air oxidation regeneration (WAOR) performed on a hydrogenation catalyst having impurities after 40 days on stream. To maintain hydrogenation conversions above or close to 95%, the inlet temperature had been increased over the first 40 days from a baseline 110° C. to just under 140° C. After the WAOR, nearly quantitative conversion was obtained at the baseline 110° C.

FIG. 3 illustrates multiple wet air oxidation regenerations performed on a hydrogenation catalyst having impurities after approximately 140 days on stream. After each WAOR, increased conversion for the hydrogenation was observed. In this example, a constant reactor temperature was used.

FIG. 4 illustrates inductively coupled plasma mass spectrometry (ICP) results from wet air oxidation regenerations performed on the hydrogenation catalyst from FIG. 3. The top results are after 90 days, and bottom results are after 115 days.

FIG. 5 illustrates two wet air oxidation regenerations performed on a fouled hydrogenation catalyst. The regeneration conditions included an operating temperature of 120° C., a reactor pressure of 100 psig, and the gas stream contained 50% air/50% N₂.

FIG. 6 illustrates three wet air oxidation regenerations performed on a fouled hydrogenation catalyst using regeneration conditions at an operating temperature of 110° C., a reactor pressure of 100 psig, and a 100% air gas stream.

FIG. 7 illustrates the CO₂ generated during a wet air oxidation performed on a fouled hydrogenation catalyst using regeneration conditions at an operating temperature of 110° C., a reactor pressure of 100 psig, and a 100% air gas stream.

### DETAILED DESCRIPTION OF THE INVENTION

Described herein are reactor systems and methods for regenerating hydrogenation catalysts for use in hydrogenating feedstock solutions, such as water-soluble sugars derived from biomass and/or unsaturated hydrocarbon streams. The provided reactor systems and methods offer unique features and advantages over existing regeneration techniques. In some embodiments, the provided reactor systems and methods for regenerating hydrogenation catalysts offer mild reaction conditions that can effectively remove impurities to restore hydrogenation catalytic activity, while additionally maintaining the catalyst's structural integrity (e.g., surface area, pore volume). Maintaining the catalyst's structural integrity and/or catalytic activity for extended periods of time improves operation economics by reducing the number of times the catalyst needs to be replaced over time and/or reducing the required frequency of regeneration operations. This is an improvement over current techniques to regenerate catalytic activity, such as hydrogen peroxide based methods, which have a tendency to degrade the catalyst's surface area and pore structure over time. Further, hydrogen peroxide poses storage challenges on a commercial scale. The regenerative oxidants provided herein are cheaper than hydrogen peroxide, and existing techniques are available to store the reagents at commercial scale.

Representative Reactor

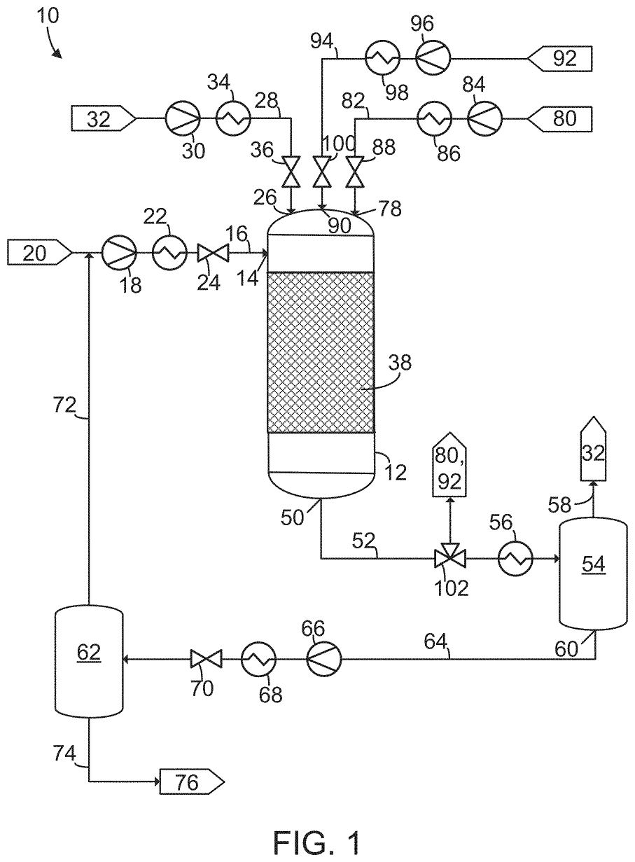

Referring to FIG. 1, a representative reactor system 10 is illustrated in accordance to some embodiments of the present disclosure. Although the principles disclosed herein can be beneficially implemented on the illustrated reactor system 10, use of other reactor system architectures is possible for some embodiments. In particular, the reactor system 10 includes a reactor 12 having a feedstock inlet 14 that places the reactor 12 in fluid communication with a feedstock conduit 16. A pump 18 may be configured in the feedstock conduit 16 to transport a feedstock solution from a feedstock source 20, such as a reservoir or upstream process unit, to the reactor 12. The feedstock conduit 16 may include a heat exchanger 22 for controlling the temperature of the feedstock solution, and a valve 24 for controlling the flow of the feedstock solution to the reactor 12.

In some embodiments, suitable feedstock solutions include water-soluble sugars derived from biomass, although other feedstocks can be used. As used herein, the term “biomass” refers to, without limitation, organic materials produced by plants (such as leaves, roots, seeds and stalks), and microbial and animal metabolic wastes. Common biomass sources include: (1) agricultural wastes, such as corn stalks, straw, seed hulls, sugarcane leavings, bagasse, nutshells, and manure from cattle, poultry, and hogs; (2) wood materials, such as wood or bark, sawdust, timber slash, and mill scrap; (3) municipal waste, such as waste paper and yard clippings; and (4) energy crops, such as poplars, willows, switch grass, alfalfa, prairie bluestream, corn, soybean, and the like. The feedstock can be fabricated from biomass by any means now known or developed in the future, or can be simply byproducts of other processes. The sugars can also be derived from wheat, corn, sugar beets, sugar cane, or molasses. The sugar is combined with water to provide an aqueous feedstock solution having a concentration effective for hydrogenating the sugar. Generally, a suitable sugar concentration is in the range of about 5% to about 70%, with a range of about 40% to 70% more common in industrial applications.

Additionally or alternatively, suitable feedstock solutions include, but are not limited to, oxygenated hydrocarbons (C₂₊O₁₊, e.g. cyclic ethers, esters, ketones, lactones, carboxylic acids), vegetable oils (e.g., polyunsaturated fatty acids), olefins (e.g., alkenes and aromatics, such as C₃-C₁₂ olefins), alkynes, aldehydes, imines, nitriles, thiols, disulfides, thioesters, thioethers, phenols, other arenes/aromatic compounds, and combinations thereof.

Referring back to FIG. 1, the reactor 12 includes a hydrogen inlet 26 that places the reactor 12 in fluid communication with a hydrogen conduit 28. A gas transport device 30 may be configured in the hydrogen conduit 28 to transport hydrogen from hydrogen source 32, such as a reservoir or upstream process unit, to the reactor 12. In some embodiments, the hydrogen conduit 28 includes a heat exchanger 34 configured to control the heat of the hydrogen stream. Suitable gas transport devices 30 include, but are not limited to, compressors or blowers. Although the hydrogen inlet 26 and the feedstock inlet 14 are orientated in a co-current direction in FIG. 1, it is to be appreciated that the hydrogen inlet 26 could be arranged in a countercurrent orientation (i.e., fed into the bottom of the reactor 12). The hydrogen conduit 28 may include a valve 36 for controlling the flow of the hydrogen to the reactor 12. Although not illustrated in FIG. 1, the feedstock and hydrogen may be blended, mixed, or otherwise combined in a mixer prior to being delivered to the reactor 12.

In some embodiments, the reactor 12 includes a hydrogenation catalyst 38 disposed therein. Hydrogenation reactions can be carried out in any reactor of suitable design, including continuous-flow, batch, semi-batch or multi-system reactors, without limitation as to design, size, geometry, flow rates, etc. The reactor system 10 can also use a fluidized catalytic bed system, a swing bed system, a fixed bed system, a moving bed system, or a combination of the above. Reactions of the present disclosure are typically practiced using a continuous flow system at steady-state equilibrium.

In some embodiments, the reactor system 10 operates as a fixed, trickle bed reactor with shell-and-tube heat exchange in which the hydrogen and feedstock solution are introduced at the top of the reactor 12 and allowed to flow downward over a fixed bed of the hydrogenation catalyst 38. The advantages of the trickle bed reactor include a simple mechanical design, a simplified operation and potentially a simplified catalyst development. The main design challenges are ensuring that the heat and mass transfer requirements of the reaction are met. The main operational challenges for trickle bed reactors are: uniformly loading the hydrogenation catalyst 38, uniformly introducing the gas and liquid feeds, and avoiding bypassing of some of the hydrogenation catalyst 38 due to channeling of the reactants as they flow through the reactor 12.

In some embodiments, the reactor system 10 operates as a slurry reactor. While a trickle bed reactor is loaded with an immobile hydrogenation catalyst 38, a slurry reactor contains a flowing mixture of reactants, products, and hydrogenation catalyst 38 particles. Keeping a uniform mixture throughout the reactor 12 includes active mixing either from a mixer or a pump. In addition, to withdraw product the catalyst particles must be separated from the product and unreacted feed by filtration, settling, centrifuging or some other means. The advantages of a slurry reactor are mainly that the active mixing might enable higher heat and mass transfer rates per unit of reactor volume.

In some embodiments, the feedstock solution and the hydrogen are reacted across the hydrogenation catalyst 38 in the reactor 12. In some embodiments, the heat exchangers 22, 34 heat the feedstock solution and hydrogen streams to a temperature from 5° C. to 700° C., from 10° C. to 500° C., from 20° C. to 300° C., or from 50° C. to 180° C. In some embodiments, the pressure of the reactor 12 is maintained from 0 psig to 5000 psig, or from 100 psig to 3000 psig. The hydrogenation catalyst 38 may be configured in the reactor 12 in various configurations including, but not limited to, a single fixed bed or in a shell and tube arrangement. In some embodiments, the reactor system 10 includes a heating system configured to provide heat to the reactor 12 to maintain a desired operating temperature. In some embodiments, the heating system provides heat to the reactor 12 using, for example, a heating element (e.g., electric heaters), a heating fluid, or combinations thereof. The heating system may be configured on the outside of the reactor. Additionally or alternatively, the heating system may be configured in a shell-and-tube configuration, where a heating fluid provides heat to the hydrogenation catalyst 38 via the shell or tube side. In some embodiments, the reactor 12 temperature can also be controlled by recycling the products of the reaction back through the reactor 12 to decrease the reaction exotherms.

The product stream exits the reactor 12 through at least one reactor outlet 50, and is optionally transported to a separator 54 via a product conduit 52. In some embodiments, the product conduit 52 includes a heat exchanger 56 to adjust the temperature of the product stream prior to entering the separator 54. The separator 54 may optionally separate unreacted hydrogen from unreacted reactants and products. The unreacted hydrogen may be recycled to the hydrogen source 32 via a hydrogen recycle conduit 58. Any suitable separator 54 may be used to separate the hydrogen from the unreacted reactants and products, including but not limited to, a settling tank, flash tank, distillation, or a combination thereof. Although not illustrated in FIG. 1, in some embodiments the reactor 12 may include a gas outlet and a liquid outlet, where the disengagement of vapor and liquid products occurs inside the reactor 12 without the separator 54.

In some embodiments, the separator 54 includes a product outlet 60 that places the separator 54 in fluid communication with a second separator 62 via conduit 64. A pump 66 may transport the product stream and unreacted reactants to the second separator 62. A heat exchanger 68 may control the temperature of the product stream and unreacted reactants entering the second separator 62, and a valve 70 may regulate the flow.

In some embodiments, the second separator 62 is configured to separate the product stream from unreacted reactants. The unreacted reactants may be recycled to the feedstock conduit 16 via recycle conduit 72, or otherwise discarded from the process. The product stream exiting the separator 62 via product conduit 74 may be sent to storage or to downstream processing units 76, such as aqueous phase reforming (APR) or hydrodeoxygenation (HDO) systems. Any suitable separator 62 may be used to separate the product stream from the unreacted reactants, including but not limited to, distillation, evaporation, liquid-liquid extraction, chromatography, or combinations thereof.

Catalyst

The present method may be used for regenerating hydrogenation catalysts, e.g., those used in the hydrogenation of biomass. In some embodiments, suitable hydrogenation catalysts 38 for the reactor system 10 includes hydrogenation catalysts 38 having an active metal and a support. Suitable active metals include, but are not limited to, Fe, Ru, Co, Pt, Pd, Ni, Re, Cu, alloys thereof, and a combination thereof, either alone or with promoters such as Ag, Au, Cr, Zn, Mn, Mg, Ca, Cr, Sn, Bi, Mo, W, B, P, and alloys or combinations thereof.

The hydrogenation catalyst may also include any one of several supports, depending on the desired functionality of the catalyst. Exemplary supports include transition metal oxides, an oxide formed from one or more metalloid, and reactive nonmetals (e.g., carbon). Non-limiting examples of supports include, but are not limited to, carbon, silica, alumina, zirconia, titania, vanadia, ceria, silica-aluminate, zeolite, kieselguhr, hydroxyapatite, zinc oxide, chromia, and mixtures thereof.

In some embodiments, the catalyst is a Ru/C hydrogenation catalyst. For example, the catalyst may comprise about 0.1% to about 5% ruthenium loaded onto a carbon particle. The catalyst may be in a form of extrudate, tablet, sphere, granule, powder, foam, a coated structure, or a combination thereof.

The catalyst may be deactivated during the reaction or chemical process it catalyzes. For example, a hydrogenation catalyst as described herein may be deactivated during biomass hydrogenation process. The catalyst may have a surface with active sites, which may affect the capacity of the catalyst in catalyzing the hydrogenation reaction. The catalyst may be deactivated due to various reasons during the hydrogenation process, including, for example, blocking of active sites by physical absorption (or deposition) of bulky molecules, poisoning of active sites by impurities in the feedstock, or a combination thereof. Catalyst poisoning may be caused by, for example, a chemical reaction or strong interaction of the impurities (e.g., sulfur containing compounds) with the active site of the catalyst, thereby lowering the capacity of the catalyst to catalyze the hydrogenation reaction, i.e., thereby deactivating the catalyst. The degree of deactivation of the catalyst may increase over time as the hydrogenation process continues. Although the amounts of impurities in the feedstock may be relatively low, at large volumes and over time the impurities can build up and adversely affect catalyst activity.

A “fresh” catalyst is used to mean a catalyst that has not been exposed to a feedstock solution or the impurities from the feedstock under hydrogenation conditions.

A “fouled hydrogenation catalyst” or “fouled catalyst” as used herein refers to a hydrogenation catalyst in which the active sites are at least partially deactivated due to being used in a hydrogenation process (i.e., exposed to a feedstock solution under conditions for hydrogenation of the feedstock solution using the catalyst). The degree of fouling may be affected, for example, by the composition of the catalyst, the duration and conditions of the hydrogenation process, the composition of the feedstock, and the amounts of impurities in the feedstock.

A “regenerated hydrogenation catalyst” or “regenerated catalyst” as used herein refers to a fouled catalyst whose catalytic capacity is at least partially restored, for example, by removing the deposits and/or accumulated impurities from the catalyst surface, restoring access to active sites, restoring poisoned active sites, or a combination thereof. As described herein, a regenerated catalyst may be re-used in a hydrogenation process and become a fouled catalyst again during the process. In this situation, the regenerated catalyst can also be referred to as a “freshly regenerated” catalyst relative to the fouled catalyst produced from such regenerated catalyst.

The catalytic capacity of a regenerated catalyst, or the catalytic capacity of a fouled catalyst from which the regenerated catalyst is produced, may be compared to that of a fresh catalyst. For example, the catalytic capacity of a fouled catalyst may be about 20%, about 30%, about 40%, about 50%, about 60%, about 70%, about 80%, or about 90% of the catalytic capacity of a fresh catalyst. For example, the catalytic capacity of a regenerated catalyst may be about 80%, about 90%, about 95%, about 99%, about 100%, or about 110% the catalytic capacity of a fresh catalyst. In some embodiments, the regenerated catalyst has more catalytic capacity than the fouled catalyst from which the regenerated catalyst is produced. For example, the regeneration method herein may restore at least a portion of the catalytic capacity in a fouled catalyst, resulting in an increase of the catalytic capacity in the regenerated catalyst. In some embodiments, the catalytic capacity in a regenerated catalyst may be about 105% to about 500% of the catalytic capacity of the fouled catalyst from which the regenerated catalyst is produced, including about 120%, about 150%, about 200%, about 300%, about 400%, or about 500%.

The catalytic capacity of a catalyst (e.g., a fresh, fouled, or regenerated catalyst) can be measured by a conversion rate of a reagent in the feedstock in a reaction (e.g., a hydrogenation reaction) that is catalyzed by such catalyst. As used herein, the term “conversion” of a hydrogenation catalyst refers to the hydrogenation catalyst's conversion over a duration (e.g., at least 1 hour to at least one day) of a reactant in the feedstock solution after being exposed to the feedstock solution for a hydrogenation cycle. The hydrogenation catalyst may be a fresh catalyst, a fouled catalyst, or a regenerated catalyst. As used herein, conversion of a specific feedstock reactant (Xᵢ) may be calculated by:

$${X}_{i} = 1 - \frac{{n}_{i} ( t )}{{n}_{i} ( t = 0 )}$$

where nᵢ is the number of moles of the specific feedstock reactant (e.g., sugar, olefin, vegetable oil, alkyne, aldehyde, imine, nitrile) at the beginning (t=0) or after a specific duration (t) of the hydrogenation process. The conversion values of a fresh catalyst, a fouled catalyst, and a regenerated catalyst may be compared under the same hydrogenation conditions (e.g., at a temperature from 50° C. to 180° C. and a pressure from 100 psig to 3000 psig), as conversion may be a function of temperature and pressure.

The “surface” or “surface area” of a catalyst as used herein includes both active surface having active sites for effective catalysis and deactivated surface with reduced catalytic capacity due to deactivation of active sites as described herein.

The active surface area of a regenerated catalyst, or the active surface area of a fouled catalyst from which the regenerated catalyst is produced, may be compared to that of a fresh catalyst as one measure of a degree of fouling (or regeneration) of a catalyst. For example, the active surface area of a fouled catalyst may be about 10%, about 20%, about 30%, about 40%, about 50%, about 60%, about 70%, about 80%, or about 90% of the active surface area of a fresh catalyst. For example, the active surface area of a regenerated catalyst may be about 80%, about 90%, about 95%, about 99%, about 100%, or about 110% the active surface area of a fresh catalyst. In some embodiments, the regenerated catalyst has more active surface area than the fouled catalyst from which the regenerated catalyst is produced. For example, the regeneration method herein may restore at least a portion of the deactivated surface in a fouled catalyst back to active surface, therefore increasing the active surface area in the regenerated catalyst. In some embodiments, the active surface area in a regenerated catalyst may be about 105% to about 500% of the active surface area of the fouled catalyst from which the regenerated catalyst is produced, including about 120%, about 150%, about 200%, about 300%, about 400%, or about 500%.

In some embodiments, the hydrogenation catalyst 38 and/or fouled hydrogenation catalyst has a total surface area from 10 m²/g to 1500 m²/g. The hydrogenation catalyst 38 may be a fresh catalyst, a fouled catalyst, or a regenerated catalyst. The retention of surface area after regeneration or use of the hydrogenation catalyst 38 may be used as an indication of the physical strength of the hydrogenation catalyst 38. In some embodiments, the hydrogenation catalyst 38 and/or fouled hydrogenation catalyst has a total surface area of at least 10 m²/g, or at least 20 m²/g, or at least 30 m²/g, or at least 40 m²/g, or at least 50 m²/g, or at least 100 m²/g, or at least 200 m²/g, or at least 300 m²/g, or at least 500 m²/g, or at least 600 m²/g, or at least 700 m²/g, or at least 800 m²/g, to less than 900 m²/g, or less than 1000 m²/g, or less than 1100 m²/g, or less than 1200 m²/g, or less than 1300 m²/g, or less than 1400 m²/g, or less than 1500 m²/g. The total surface area of the pores may be measured using, for example, adsorption based methods such as Brunauer-Emmet-Teller nitrogen or argon adsorption, or other suitable techniques.

In some embodiments, the hydrogenation catalyst 38 comprises micropores and mesopores. The hydrogenation catalyst 38 may be a fresh catalyst, a fouled catalyst, or a regenerated catalyst. As used herein, the term “micropore” refers to pores in the hydrogenation catalyst 38 that have a pore diameter of less than 2 nm. As used herein, the term “mesopore” refers to pores in the hydrogenation catalyst 38 that have a pore diameter from 2 nm to 50 nm.

In some embodiments, the hydrogenation catalyst 38 has a micropore surface area of at least 5 m²/g, or at least 10 m²/g, or at least 20 m²/g, or at least 30 m²/g, or at least 50 m²/g, or at least 100 m²/g, at least 100 m²/g, or at least 200 m²/g, or at least 300 m²/g, or at least 500 m²/g, or at least 600 m²/g, or at least 700 m²/g, or at least 800 m²/g, to less than 900 m²/g, or less than 1000 m²/g, or less than 1100 m²/g, or less than 1200 m²/g, or less than 1300 m²/g, or less than 1400 m²/g, or less than 1450 m²/g. The micropore surface area of the pores may be measured using, for example, adsorption based methods such as Brunauer-Emmet-Teller nitrogen or argon adsorption, or other suitable techniques. The micropore surface area may be determined by following the IUPAC guidelines provided in Thommes et al. Pure Appl. Chem. 2015, “Physisorption of gases, with special reference to the evaluation of surface area and pore size distribution (IUPAC Technical Report)”.

In some embodiments, the hydrogenation catalyst 38 has a mesopore surface area of at least 0.5 m²/g, or at least 1 m²/g, or at least 2 m²/g, or at least 3 m²/g, or at least 5 m²/g, or at least 10 m²/g, or at least 20 m²/g, or at least 30 m²/g or at least 40 m²/g, to less than 50 m²/g, or less than 60 m²/g, or less than 70 m²/g, or less than 80 m²/g, or less than 90 m²/g, or less than 100 m²/g, or less than 110 m²/g, or at least 125 m²/g, or at least 150 m²/g. The mesopore surface area of the pores may be measured using, for example, adsorption based methods such as Brunauer-Emmet-Teller nitrogen or argon adsorption, or other suitable techniques. The mesopore surface area may be determined by following the IUPAC guidelines provided in Thommes et al. Pure Appl. Chem. 2015, “Physisorption of gases, with special reference to the evaluation of surface area and pore size distribution (IUPAC Technical Report)”.

In some embodiments, the reactor system 10 includes pre-treatment units or steps to process the feedstock solution and/or hydrogenation catalyst 38. For example, the hydrogenation catalyst 38 may be reduced into an active state. For example, during production, the catalyst can be reduced and, in certain applications, then passivated with low levels of oxygen to stabilize the catalyst when exposed to air. The purpose of the reduction step is to transform any oxidized catalyst into a fully reduced state. For certain feedstock solutions, a pre-treatment step may be included upstream of the reactor system 10. For example, sugars containing glycosidic bonds (e.g., sucrose) may be hydrolyzed prior to hydrogenation in the reactor 12.

Catalyst Regeneration

During hydrogenation, catalyst impurities may build up on the surface of the hydrogenation catalyst 38 and reduce catalytic performance. As used herein, the terms “catalyst impurity” or “impurity” refers to impurities that form deposits that accumulate on catalytic sites on the surface of hydrogenation catalyst 38, restrict access to the catalytic sites, and/or reduce catalytic activity over time (i.e., results in lower conversion and yields of products). Exemplary catalyst impurities include, but are not limited to, carbon-containing impurities, sulfur-containing impurities, silicon-containing impurities, phosphorus-containing impurities, or iron-containing impurities.

In some embodiments, the hydrogenation catalyst 38 is regenerated into a regenerated catalyst by contacting the hydrogenation catalyst 38 with a flushing medium. In some embodiments, the flushing medium comprises a vapor phase and a liquid phase.

Still referring to FIG. 1, the reactor 12 includes a vapor phase inlet 78 that places the reactor 12 in fluid communication with a vapor phase source 80 via vapor phase conduit 82. A fluid transport device 84 (e.g., compressor or blower) may be configured in the vapor phase conduit 82 to transport the vapor phase from the vapor phase source 80 to the reactor 12. The vapor phase conduit 82 may include a heat exchanger 86 for controlling the temperature of the flushing medium's vapor phase, and a valve 88 for controlling the flow of the vapor phase to the reactor 12. In some embodiments, the fluid transport device 84 is configured for direct air or atmospheric capture, where the fluid transport device 84 is in fluid communication or in direct fluid communication with atmospheric air for compression. Using air as the vapor phase in the flushing medium offers various advantages. Specifically, this would avoid having to purchase and store other oxidants (e.g., hydrogen peroxide) on site. In some embodiments, the vapor phase source 80 includes an inert gas (e.g. nitrogen, argon, helium, neon, krypton, xenon, radon, or combinations thereof) source and oxygen source (e.g., compressed tank) that may be used to alter the O₂ and/or inert gas content of the vapor phase to the concentrations described herein.

In some embodiments, the reactor 12 includes a liquid phase inlet 90 that places the reactor 12 in fluid communication with a liquid phase source 92 via liquid phase conduit 94. A pump 96 may be configured in the liquid phase conduit 94 to transport the liquid phase from the liquid phase source 92 to the reactor 12. The liquid phase conduit 94 may include a heat exchanger 98 for controlling the temperature of the flushing medium's liquid phase, and a valve 100 for controlling the flow of the vapor phase to the reactor 12. Although not illustrated in FIG. 1, the liquid phase and vapor phase may be blended, mixed, or otherwise combined in a mixer prior to being delivered to the reactor 12.

In some embodiments, the regenerated hydrogenation catalyst may be produced by maintaining contact of the flushing medium with the hydrogenation catalyst 38 at a regeneration temperature, a regeneration pressure, and a duration sufficient to remove at least a portion of the impurities from the hydrogenation catalyst 38. Contacting the flushing medium to the hydrogenation catalyst 38 may occur in any suitable flow scheme, including continuous flow of flushing medium over the hydrogenation catalyst 38 without recycle, continuous flow of flushing medium over the hydrogenation catalyst 38 with some or full recycle, batch, or semi-batch flow. In some embodiments, the flushing medium exits the reactor 12 through reactor outlet 50, and is recycled to the flushing medium sources 80, 92 or reactor inlets 78, 90 by controlling the flow in product conduit 52 with valve 102.

In some embodiments, the regeneration temperature is from 50° C. to 200° C. In some embodiments, the regeneration temperature is at least 50° C., or at least 60° C., or at least 70° C., or at least 80° C., or at least 90° C., or at least 100° C., or at least 110° C., or at least 120° C., or at least 130° C., to less than 140° C., or less than 150° C., or less than 160° C., or less than 170° C., or less than 180° C., or less than 190° C., or less than 200° C.

In some embodiments, the regeneration pressure is from 20 psig to 300 psig. In some embodiments, the regeneration pressure is at least 20 psig, or at least 30 psig, or at least 40 psig, or at least 50 psig, or at least 60 psig, or at least 70 psig, or at least 80 psig, or at least 90 psig, or at least 100 psig, to less than 110 psig, or less than 125 psig, or less than 150 psig, or less than 200 psig, or less than 250 psig, or less than 300 psig.

In some embodiments, the duration of contacting the flushing medium to the hydrogenation catalyst 38 is from 10 minutes to one week, or from 30 minutes to 24 hours, or from 1 hour to 12 hours. In some embodiments, the duration of contacting the flushing medium to the hydrogenation catalyst occurs for at least 30 minutes, or at least 1 hour, or at least 2 hours, or at least 3 hours, or at least 4 hours, or at least 5 hours, at least 6 hours, or to less than 12 hours, or less than 24 hours, or less than 2 days, or less than 3 days, or less than 4 days, or less than 5 days, or less than 6 days, or less than one week, or longer.

In some embodiments, the oxygen flow to the reactor can be stopped while the liquid flushing medium is continued. The duration of the extra liquid flushing occurs for at least 30 minutes, or at least 1 hour, or at least 2 hours, or at least 3 hours, or at least 4 hours, or at least 5 hours, at least 6 hours, or less than 24 hours, or less than 2 days, or less than 3 days, or less than 4 days, or less than 5 days, or less than 6 days, or less than one week, or longer.

In some embodiments, the flushing medium comprises water, oxygen, and an inert gas. In some embodiments, the vapor phase has an oxygen content from 0.5% (v/v) to 60% (v/v), from 1% (v/v) to 50% (v/v), or from 5% (v/v) to 30% (v/v). In some embodiments, the vapor phase has an oxygen content of at least 0.5% (v/v), or at least 1%, or at least 5%, or at least 10%, or at least 15%, or at least 20%, or at least 25%, or at least 30%, or at least 35%, to less than 40%, or less than 45%, or less than 50%, or less than 55%, or less than 60%.

In some embodiments, the vapor phase of the flushing medium has an inert gas content from 40% (v/v) to 99.5% (v/v). In some embodiments, the vapor phase has an inert gas content of at least 40% (v/v), or at least 45%, or at least 50%, or at least 55%, or at least 60%, or at least 65%, or at least 70%, or at least 75%, or at least 80%, to less than 85%, or less than 90%, or less than 95%, or less than 99.5% (v/v).

In some embodiments, the vapor phase is composed of air. As used herein, “air” may refer to gases surrounding the earth, which may vary regionally, and are a function of various factors, such as temperature and pressure. As one example, the term “air” may refer to a gaseous composition composed, in a dry volume percentage (vol %), of about 78 vol % nitrogen, about 20.9 vol % oxygen, about 0.9 vol % argon, about 0.04 vol % carbon dioxide, and other elements and compounds such as helium, methane, krypton, hydrogen, nitrous oxide, xenon, ozone, carbon monoxide, sulfur dioxide, nitrogen dioxide, and ammonia.

In some embodiments, the oxygen content in the flushing medium is based on the amount of hydrogenation catalyst 38 in the reactor 12. In some embodiments, the flushing medium comprises an oxygen to catalyst flux ratio (O₂/cat/hr) from 0.1*10⁻³ to 100*10⁻³ (mol/g/hr). In some embodiments, the O₂/cat/hr flux ratio is at least 0.1*10⁻³ (mol/g/hr), or at least 0.5*10⁻³, or at least 1*10⁻³, to less than 5*10⁻³, or less than 10*10⁻³, or less than 50*10⁻³, or less than 100*10⁻³ (mol/g/hr).

In some embodiments, the water content in the flushing medium is based on the amount of hydrogenation catalyst 38 in the reactor 12. In some embodiments, the flushing medium comprises a water to catalyst flux ratio (H₂O/cat/hr) from 1 to 100 (g/g/hr). In some embodiments, the H₂O/cat/hr ratio is at least 1, or at least 2, or at least 5, or less than 10, or less than 20, or less than 100 (g/g/hr).

The inclusion of water in the flushing medium offers various advantages. First, water acts as a heat sink in the flushing medium that allows improved control over the temperature of the reactor 12 relative to a flushing medium composed solely of gases. This improved heat control avoids the creation of hot spots that may burn away catalytic supports, such as carbon. Water is also a polar solvent that may facilitate the removal of certain impurities, such as ionic salts and other polar moieties. Second, the inclusion of water in the flushing medium allows the hydrogenation catalyst 38 to remain wetted during regeneration. Flushing media composed solely of gases can dry out the catalyst, which can create cracks in the fixed bed and lead to an increase in replacement frequency.

In some embodiments, the flushing medium is substantially free or entirely free of hydrogen peroxide. As used herein, the term “substantially free” refers to less than 1%, or less than 0.5%, or less than 0.1%, or less than 0.05% hydrogen peroxide. In some embodiments, the flushing medium is substantially free or entirely free of hydrogen peroxide prior to entering the reactor 12.

Unlike typical catalyst regeneration processes, which operate in gas-phase conditions under temperatures in excess of 200° C. (e.g., decoking and desulphurization reactions), or utilize oxidants that degrade the catalyst's physical structure over time (e.g., H₂O₂-based regeneration), the present disclosure provides a method for regenerating a hydrogenation catalyst 38 with a flushing medium that operates under less severe conditions (e.g., temperatures of less than 200° C.). Surprisingly and unexpectedly, a flushing medium comprising water, oxygen, and an inert/diluent gas at the specified regeneration pressures and temperatures is effective in restoring catalytic activity by removing a sufficient amount of impurities from the hydrogenation catalyst 38 to restore catalytic activity. Further, it was surprisingly and unexpectedly found that a flushing medium comprising water, oxygen, and nitrogen is effective in maintaining the fouled hydrogenation catalyst's 38 activity (e.g., conversion efficacy) and structural integrity (e.g., total surface, pore size, pore volume) after contacting the flushing medium over the specified duration.

In some embodiments, the term fouled hydrogenation catalyst refers to a hydrogenation catalyst 38 that has been exposed to a feedstock solution under the specified hydrogenation conditions (e.g., temperatures, pressures, concentrations of feedstock) described herein for a period of time (e.g., at least 1 day, at least 2 days, at least 3 days, at least 4 days, at least 5 days, at least 6 days, at least one week, at least two weeks, at least three weeks, at least one month, at least six months, at least one year). The fouled hydrogenation catalyst can be produced by exposing a fresh catalyst that has never been exposed to the feedstock solution at the specified hydrogenation conditions, or by exposing a freshly regenerated catalyst to the specified hydrogenation conditions.

In some embodiments, the regenerated hydrogenation catalysts exhibit improved structural integrity and/or catalytic activity relative to catalysts regenerated using hydrogen peroxide-based regeneration. In some embodiments, upon regeneration using the provided methods, the provided regenerated hydrogenation catalysts are characterized by retaining at least a portion of the total surface area of the fouled hydrogenation catalyst (e.g., at least 70%, or at least 75%, or at least 80%, or at least 85%, or at least 90%, or at least 95%, or at least 96%, or at least 97%, or at least 98% or at least 99% of the value of the fouled hydrogenation catalyst) after contacting the flushing medium for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week).

As used herein, the term “retain,” “retaining,” or “retention,” with respect to a reference value, includes both partial and increased values relative to the reference value. For example, a specified parameter (e.g., surface area or conversion of a regenerated catalyst) can retain less than 100% or more than 100% of a reference parameter (e.g., surface area or conversion of a fouled catalyst from which the regenerated catalyst is produced).

In some embodiments, the regenerated hydrogenation catalysts retain surface area after exposure to multiple regeneration cycles. As used herein, the term “multi-regenerated hydrogenation catalyst,” refers to a hydrogenation catalyst that has been exposed to multiple hydrogenation cycles under one or more of the provided hydrogenation conditions, and multiple regenerations under one or more the provided regeneration conditions. With regard to catalysts that are multi-regenerated hydrogenation catalysts, discussion of the retention of surface area following regeneration refers to a comparison of surface area for the fouled hydrogenation catalyst before a given regeneration (e.g., before a second regeneration, a third regeneration, a fourth regeneration, a fifth regeneration, a sixth regeneration, a seventh regeneration, an eighth regeneration, a ninth regeneration, a tenth regeneration, etc.) relative to the surface area immediately following the given regeneration. In some embodiments, upon multiple regenerations using the provided methods, the provided multi-regenerated hydrogenation catalysts are characterized by retaining at least a portion of the total surface area of the fouled hydrogenation catalyst at the given regeneration cycle (e.g., at least 70%, or at least 75%, or at least 80%, or at least 85%, or at least 90%, or at least 95%, or at least 96%, or at least 97%, or at least 98% or at least 99% of the value of the total surface area of the fouled hydrogenation catalyst) after contacting the flushing medium for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week).

In some embodiments, upon regeneration using the provided methods, the provided regenerated hydrogenation catalysts are characterized as regaining at least a portion of the total surface area of the fouled hydrogenation catalyst (e.g., gain at least 1%, or at least 2%, or at least 3%, or at least 4%, or at least 5%) after contacting the flushing medium for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week).

In some embodiments, the provided methods are effective at removing impurities from the hydrogenation catalyst 38. In some embodiments, upon regeneration using the provided methods, the provided regenerated hydrogenation catalysts have a reduction in an impurity (e.g., at least a 5% reduction, or at least a 10% reduction, or at least a 15% reduction, or at least a 20% reduction, or at least a 30% reduction, or at least a 40% reduction, or at least a 50% reduction, or at least a 60% reduction) relative to the impurity content of the fouled hydrogenation catalyst after contacting the flushing medium to the hydrogenation catalyst 38 for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week). In some embodiments, the reduction in the impurity may be obtained by sampling the fouled hydrogenation catalyst to obtain an initial impurity content. The same procedure may be performed on the regenerated hydrogenation catalyst, and the results may be compared to determine the percent reduction in the impurity. In some embodiments, the content of the impurity may be obtained through any known method, such as inductively coupled plasma mass spectrometry (ICP analysis). In some embodiments, the impurity is a sulfur-containing species. In some embodiments, the removal of carbonaceous deposits can be measured by monitoring the amount of CO₂ in the effluent gas.

In some embodiments, the regenerated hydrogenation catalysts exhibit excellent retention in catalytic activity after regenerative treatment. In some embodiments, upon regeneration using the provided methods, the provided regenerated hydrogenation catalysts are characterized as retaining a portion of the conversion of the fouled hydrogenation catalysts for the feedstock solution (e.g., at least 70% of the fouled hydrogenation catalyst's conversion of the feedstock solution, or at least 75%, or at least 80%, or at least 85%, or at least 90%, or at least 95%, or at least 96%, or at least 97%, or at least 98%, or at least 99%) after contacting the flushing medium for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week).

In some embodiments, the regenerated hydrogenation catalysts retain conversion after exposure to multiple regeneration cycles. With regard to catalysts that are multi-regenerated hydrogenation catalysts, discussion of the retention of conversion following regeneration refers to a comparison of conversion for the fouled hydrogenation catalyst before a given regeneration (e.g., before a second regeneration, a third regeneration, a fourth regeneration, a fifth regeneration, a sixth regeneration, a seventh regeneration, an eighth regeneration, a ninth regeneration, a tenth regeneration, etc.) relative to the conversion immediately following the given regeneration. In some embodiments, upon multiple regenerations using the provided methods, the provided multi-regenerated hydrogenation catalysts are characterized as retaining a portion of the conversion of the fouled hydrogenation catalysts for the feedstock solution (e.g., at least 70% of the fouled hydrogenation catalyst's conversion of the feedstock solution, or at least 75%, or at least 80%, or at least 85%, or at least 90%, or at least 95%, or at least 96%, or at least 97%, or at least 98%, or at least 99%) after contacting the flushing medium for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week).

Typically, the fouled catalyst has lower catalytic capacity (e.g., as measured by the conversion value) than the fresh catalyst. The catalytic capacity of a fouled catalyst may be increased by the regeneration method as described herein to a level close to that of the fresh (or freshly regenerated) catalyst from which the fouled catalyst is produced. That is, the regeneration method herein may be used to retore the catalytic capacity of a fouled catalyst back to the level of the fresh (or freshly regenerated) catalyst. The conversion value as describe herein (as a measurement of catalytic capacity) of a regenerated catalyst, or the conversion value of a fouled catalyst from which the regenerated catalyst is produced, may be compared to that of a fresh catalyst. For example, the conversion value of a fouled catalyst may be about 50%, about 60%, about 70%, about 80%, or about 90% of the conversion value of a fresh catalyst. For example, the conversion value of a regenerated catalyst may be about 70%, about 80%, about 90%, about 95%, about 99%, about 100%, or about 110% the catalytic capacity of a fresh catalyst. In some embodiments, the conversion value of a regenerated catalyst is at least 5%, at least 10%, at least 20%, at least 50%, at least 70%, at least 90%, or at least 100% higher than that of a fouled catalyst. In some embodiments, regenerated catalyst retains at least 100%, at least 105%, at least 110%, at least 120%, at least 150%, at least 170%, at least 190%, or at least 200% of the conversion value of a fouled catalyst. As an example, the conversion value of a fresh catalyst is 0.96 and the conversion value of a fouled catalyst is 0.70 (or 73% of the fresh catalyst). After regeneration, the conversion value of the regenerated catalyst is 0.94 (or 98% of the fresh catalyst). In this example, the regenerated catalyst retains 134% of the conversion of the fouled catalyst (or, the conversion value of a regenerated catalyst is 34% higher than that of the fouled catalyst).

In some embodiments, the regenerated hydrogenation catalyst provided herein exhibits improved structural integrity through the retention of micropore surface area relative to catalysts regenerated with hydrogen peroxide-based regeneration. In some embodiments, upon regeneration using the provided methods, the provided regenerated hydrogenated catalysts are characterized as retaining at least a portion of the micropore surface area (e.g., at least 70%, or at least 75%, or at least 80%, or at least 85%, or at least 90%, or at least 95%, or at least 96%, or at least 97%, or at least 98% or at least 99%) of the fouled hydrogenation catalyst after contacting the flushing medium for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week).

In some embodiments, the regenerated hydrogenation catalyst exhibits improved structural integrity through the retention of mesopore surface area relative to catalysts regenerated with hydrogen peroxide-based regeneration. In some embodiments, upon regeneration using the provided methods, the provided regenerated hydrogenated catalysts are characterized as retaining at least a portion of the mesopore surface area (e.g., at least 70%, or at least 75%, or at least 80%, or at least 85%, or at least 90%, or at least 95%, or at least 96%, or at least 97%, or at least 98% or at least 99%) of the fouled hydrogenation catalyst after contacting the flushing medium for a period of time (e.g., at least 30 minutes, or at least 1 hour, or at least 2 hours, or at least 3 hours, or at least 4 hours, or at least 5 hours, at least 6 hours, or at least 12 hours, or at least 24 hours, or at least 2 days, or at least 3 days, or at least 4 days, or at least 5 days, or at least 6 days, or at least one week).

In some embodiments, the provided reactor systems and methods provided herein offer advantages over existing regeneration techniques and catalysts. In some embodiments, the provided reactor systems and methods for regenerating hydrogenation catalysts 38 offer mild reaction conditions that can effectively remove impurities to restore hydrogenation catalytic activity, while additionally maintaining the catalyst's structural integrity (e.g., surface area, pore volume). Maintaining the catalyst's structural integrity and/or catalytic activity for extended periods of time improves operation economics by reducing the number of times the catalyst needs to be replaced over time and/or reducing the required frequency of regeneration operations. This is an improvement over current techniques to regenerate catalytic activity, such as hydrogen peroxide based methods, which have a tendency to degrade the catalyst's surface area and pore structure over time. Further, hydrogen peroxide poses storage challenges on a commercial scale. The regenerative oxidants provided herein are cheaper than hydrogen peroxide, and existing techniques are available to store the reagents at commercial scale. Further, the provided flushing medium allows improved temperature control of the reactor 12 relative to flushing mediums composed solely of gases. In some embodiments, the flushing medium may include a vapor phase that is composed of atmospheric air, which may be obtained by direct air capture. This avoids having to purchase and store chemicals, such as hydrogen peroxide, on site thereby reducing operation costs and improving plant economics.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

The present invention has been described in terms of one or more preferred embodiments, and it should be appreciated that many equivalents, alternatives, variations, and modifications, aside from those expressly stated, are possible and within the scope of the invention.

### EXAMPLES

### Example 1

Regeneration tests on hydrogenation catalysts having impurities were conducted using a flushing medium composed of water, nitrogen, and oxygen. The process was termed “wet air oxidation regeneration” (WAOR). Fresh Ru/C hydrogenation catalysts were deactivated by hydrogenating dextrose monohydrate or corn syrup feeds. The hydrogenation reactions were operated on stream for multiple days until the hydrogenation catalysts were deactivated. WAOR experiments were performed on deactivated hydrogenation catalysts according to the conditions in Table 1. The flushing medium included a liquid phase comprising water, and a vapor phase comprising nitrogen and oxygen.

TABLE 1
Conditions of WAORs conducted in various reactor systems.

| | Run 1 | Run 2 | Run 3 | Run 4 |
|---|---|---|---|---|
| Reactor OD (in) | 0.5 | 0.5 | 1 | 2 |
| Catalyst Charge (g) | 13.8 | 13 | 93.6 | 4257 |
| Oxygen % | 6.4 | 1.2 | 1 | 1 |
| Vapor Flow (NL/h) | 23.2 | 127.2 | 96.0 | 9590 |
| Liquid Flow (g/h) | 456 | 420 | 336 | 35000 |
| Temperature (° C.) | 140 | 140 | 140 | 140 |
| Reactor Press. (psig) | 75 | 50 or 75 | 75 | 100 |
| Duration (hour) | 4 | 24 | 24 | 24 |
| O₂/(cat*hr) (mol*g⁻¹*hr⁻¹) | 4.7*10⁻³ | 6.9*10⁻³ | 0.48*10⁻³ | 1.1*10⁻³ |
| H₂O/(cat*hr) (g*g⁻¹*hr⁻¹) | 33.0 | 32.3 | 3.6 | 8.2 |

All of the Runs 1-4 were successful in restoring the catalyst's activity and maintained the structural integrity of the catalyst.

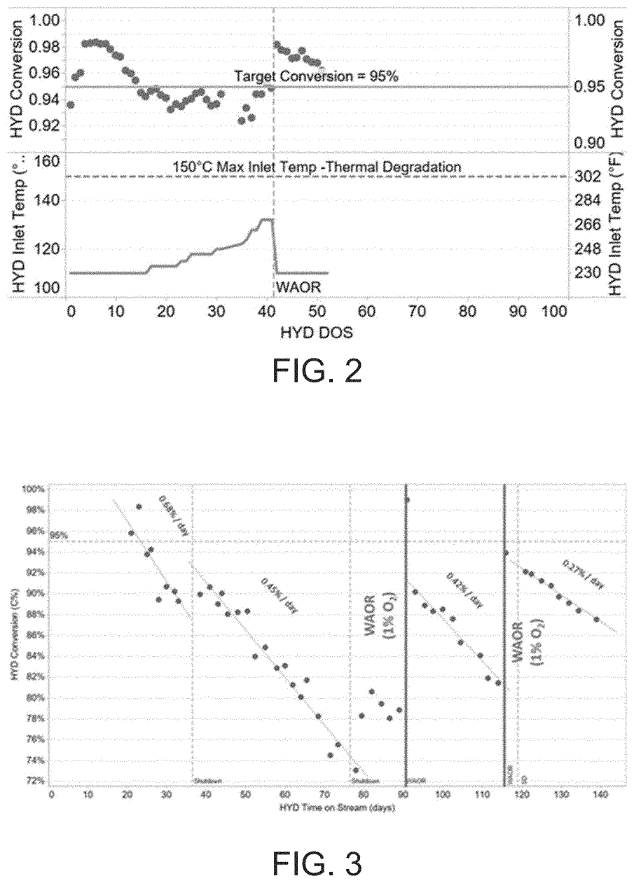

Referring to FIG. 2, Run 4 demonstrated a glucose conversion of approximately 9800 at a reactor inlet temperature of 110° C. was obtained with a catalyst that, prior to regeneration, displayed a conversion of approximately 9400 at a reactor inlet temperature of 132° C. (FIG. 2). A higher temperature had been used pre-regeneration in an attempt to maintain the glucose conversion >95%. High yields at the lower temperature post-regeneration indicate catalyst regeneration was successful.

In addition, multiple regenerations were performed on the regenerated hydrogenation catalyst from Run 3, and each time recovery of activity was observed (FIG. 3). For example, after the first regeneration, the regenerated hydrogenation catalyst had a conversion of approximately 90%, which is 93.8% of the activity of the fresh hydrogenation catalyst (activity of 960%). After the second regeneration, the regenerated hydrogenation catalyst had a conversion of approximately 92% (i.e. the regenerated hydrogenation catalyst retained 102% of the activity of the freshly regenerated catalyst and 96% of the fresh hydrogenation catalyst).

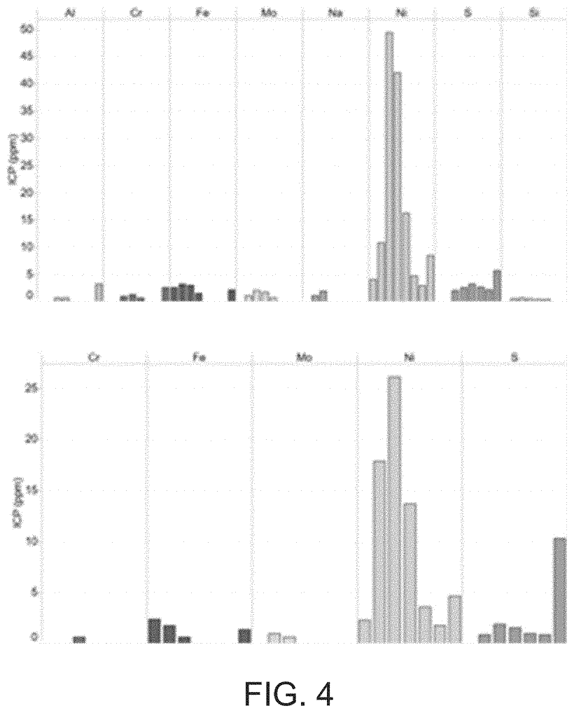

FIG. 4 shows the elemental compositions of the aqueous streams from multiple samples taken during the two regenerations from Run 3. Sulfur is detected in the aqueous product. The bars represent samples taken before introduction of 02, 30 minutes into the regeneration, 1 hour into the regeneration, 2 hours into the regeneration, 4 hours into the regeneration, approximately 18 hours into the regeneration, and 24 hours into the regeneration. Overall, 0.024 g of sulfur was removed in the first regeneration, and 0.015 g of sulfur was removed in the second regeneration.

The catalyst was unloaded at the end of Run 3 and had a sulfur concentration of 579 ppm, as measured by ICP. A portion of this catalyst was loaded into a reactor and regenerated under the conditions for Run 5 (see below). The regenerated catalyst was unloaded in four sections, and the sulfur concentration in each of the four sections is given in Table 2. The concentration of sulfur for each catalyst section is below the value for the loaded catalyst, further supporting the removal of sulfur during the catalyst regeneration.

TABLE 2

| | Catalyst section | | | |
|---|---|---|---|---|
| | 1/4 (top) | 2/4 | 3/4 | 4/4 (bottom) |
| S concentration (ppm) | 403 | 329 | 562 | 460 |

In addition to measuring the sulfur concentration by ICP, the total surface area of the fresh, fouled, and regenerated hydrogenation catalysts were measured using Ar as the adsorptive gas (Table 3). The fouled hydrogenation catalyst had a lower total surface area, micropore area and volume, and mesopore area and volume than the fresh catalyst. After regeneration, the total surface area, micropore area, micropore volume, and mesopore area all increased. Without being limited to any particular theory, it is hypothesized that the reduction of total surface area, micropore area and volume, and mesopore area and volume in the fouled catalyst results from molecule deposit, impurity accumulation, active site poisoning (e.g., by impurities), or a combination thereof on the surface of the catalyst during hydrogenation. These data demonstrate that the present regeneration method may restore the total surface area, micropore area and volume, and/or mesopore area and volume of a fouled hydrogenation catalyst, for example, by removing the deposit and/or impurities from the surface of the catalyst.

TABLE 3

| | | Total | | | | |
|---|---|---|---|---|---|---|
| | | Surface | Micropore | | Mesopore | |
| | | Area | Area | Volume | Area | Volume |
| Catalyst | | (m²/g) | (m²/g) | (cm³/g) | (m²/g) | (cm³/g) |
| Fresh | | 1188.8 | 1124.2 | 0.43 | 67.1 | 0.06 |
| Fouled | | 900.2 | 840.7 | 0.31 | 61.0 | 0.05 |
| Re- | Section 1/4 | 1007.9 | 941.0 | 0.35 | 68.6 | 0.06 |
| generated | (top) | | | | | |
| | Section 2/4 | 893.2 | 834.0 | 0.31 | 58.5 | 0.05 |
| | Section 3/4 | 938.7 | 876.9 | 0.33 | 62.3 | 0.05 |
| | Section 4/4 | 900.9 | 830.7 | 0.31 | 62.7 | 0.05 |
| | (bottom) | | | | | |
| | Average | 935.2 | 870.7 | 0.33 | 63.0 | 0.05 |

A test was performed on a catalyst from Run 2 where the catalyst underwent “regeneration” without being exposed to feed. This test would help determine whether the regeneration conditions damaged the catalyst and/or catalyst support. This catalyst displayed good physical strength properties after being regenerated and unloaded from the reactor. Specifically, Table 4 compares the total surface area of the fresh hydrogenation catalyst to the regenerated hydrogenation catalyst after 24 hours of regeneration. Four regenerated hydrogenation samples were taken from the reactor indicated by “Regen sample-1” through “Regen sample-4” in Table 4, with “Regen sample-1” coming from the top of the reactor and “Regen sample-4” coming from the bottom of the reactor.

TABLE 4

| | Total | Micropore | | Mesopore | |
|---|---|---|---|---|---|
| | Surface Area | Area | Volume | Area | Volume |
| Run 4 Catalyst | (m²/g) | (m²/g) | (cm³/g) | (m²/g) | (cm³/g) |
| Fresh Catalyst | 1169.36 | 1095.36 | 0.41 | 66.25 | 0.06 |
| Regen sample-1 | 1188.54 | 1108.57 | 0.42 | 73.06 | 0.06 |
| Regen sample-2 | 1131.91 | 1059.66 | 0.40 | 67.11 | 0.06 |
| Regen sample-3 | 1083.77 | 1011.021 | 0.38 | 67.86 | 0.06 |
| Regen sample-4 | 1041.58 | 974.14 | 0.37 | 70.07 | 0.06 |
| Regen average | 1111.45 | 1038.35 | 0.39 | 69.53 | 0.06 |

As shown in Table 4, the regenerated hydrogenation catalyst on average retained 95% of the fresh hydrogenation catalysts total surface area. Some of the regeneration hydrogenation catalysts had total surface areas that exceeded the fresh hydrogenation catalyst's total surface area (e.g., Regen sample-1). It is hypothesized that the increase of surface area may result from limited removal of existing carbon support, opening up further micropores in the catalyst structure. Overall, the regenerated hydrogenation catalysts exhibit excellent retention of physical integrity.

### Example 2

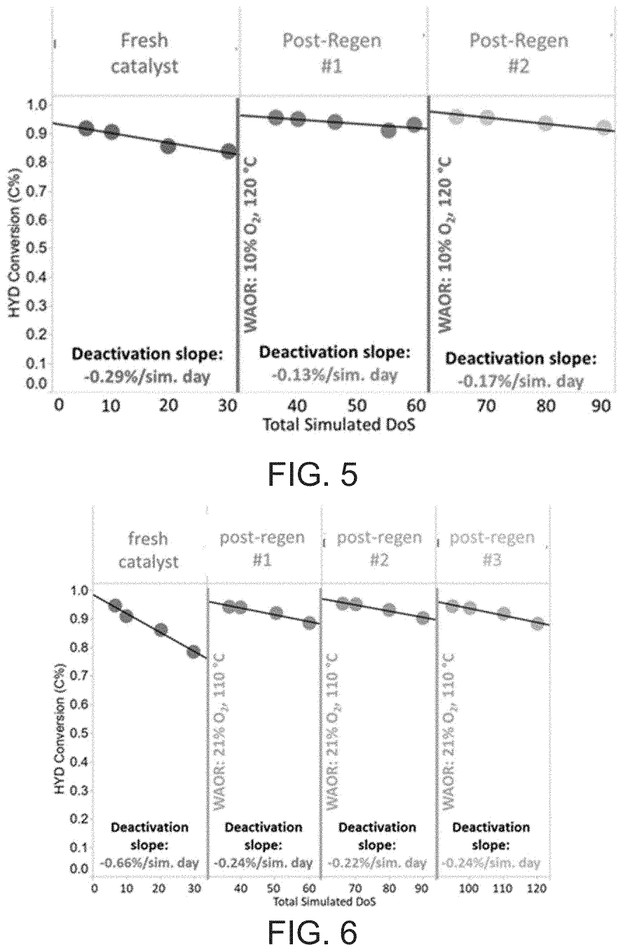

Hydrogenation catalysts having impurities were subjected to regeneration methods to restore catalytic activity. The first condition selected was 110° C. and 250 mL/min air flow, and the second condition was 120° C. and 400 mL/min gas flow of a 50:50 air/N₂ mix. The 120° C./50% air conditions were successful in regenerating catalyst activity over two cycles (FIG. 5). Conversion >95% were obtained after each regeneration. Similar results were obtained over three cycles with the 110° C./100% air strategy (FIG. 6).

TABLE 5
Conditions of WAORs conducted in various
reactor systems using higher air ratios.

| | Run 5 | Run 6 |
|---|---|---|
| Reactor OD (in) | 0.5 | 0.5 |
| Catalyst Charge (g) | 13 | 13 |
| Oxygen % | 21 | 11.0 |
| Vapor Flow (NL/h) | 15 | 24 |
| Liquid Flow (g/h) | 420 | 420 |
| Temperature (° C.) | 110 | 120 |
| Reactor Press. (psig) | 100 | 100 |
| Duration (hour) | 24 | 24 |
| O₂/(cat*hr) | 10.8*10⁻³ | 9.1*10⁻³ |
| (mol*g⁻¹*hr⁻¹) | | |
| H₂O/(cat*hr) | 32.3 | 32.3 |
| (g*g⁻¹*hr⁻¹) | | |

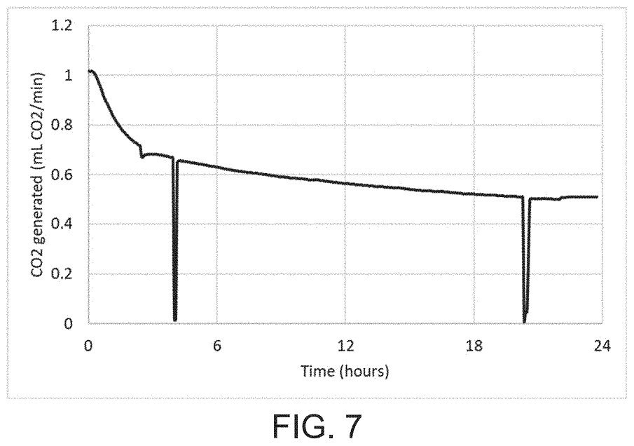

FIG. 7 shows the amount of CO₂ in the effluent gas from one of the regenerations conducted as part of Run 5. The decreasing concentration of CO₂ indicates the removal of carbonaceous deposits from the catalyst.

Due to the reasonably mild conditions employed, this regeneration is applicable to a wide array of technologies. Due to lower reaction temperatures, catalyst sintering will likely be minimized, when compared to other high temperature regeneration approaches, such as hot hydrogen stripping. Avoiding the use of mineral acids alleviates on-site storage concerns since the only inputs needed are water and air (and potentially N₂).

For reasons of completeness, various aspects of the invention are set out in the following numbered clauses:

Clause 1. A method for producing a regenerated hydrogenation catalyst from a fouled hydrogenation catalyst, the fouled hydrogenation catalyst having a total surface area and at least one associated impurity, the method comprising:

maintaining contact between the fouled hydrogenation catalyst and a flushing medium that comprises water, oxygen, and an inert gas at a regeneration temperature and a regeneration pressure sufficient to remove at least a portion of the at least one impurity from the hydrogenation catalyst to produce the regenerated hydrogenation catalyst,

wherein the regenerated hydrogenation catalyst is characterized as retaining at least 70% of the total surface area of the fouled hydrogenation catalyst.

Clause 2. The method of clause 1, wherein the regenerated hydrogenation catalyst is characterized as retaining at least 70% of the total surface area of the fouled hydrogenation catalyst after contacting the flushing medium to the fouled hydrogenation catalyst for at least 1 hour, or at least 6 hours, or at least 12 hours, or at least 24 hours, or at least 2 days, or at least one week at the regeneration temperature and the regeneration pressure.

Clause 3. The method of clause 1 or 2, wherein the regenerated hydrogenation catalyst is characterized as retaining at least 80%, or at least 90%, or at least 95% of the total surface area of the fouled hydrogenation catalyst after contacting the flushing medium to the fouled hydrogenation catalyst for at least 1 hour at the regeneration temperature and the regeneration pressure.

Clause 4. The method of any one of the preceding clauses, wherein the regenerated hydrogenation catalyst is characterized as exhibiting at least a 5% reduction in an impurity relative to the fouled hydrogenation catalyst.

Clause 5. The method of clause 4, wherein the impurity is a sulfur-containing impurity.

Clause 6. The method of clause 4, wherein the regenerated hydrogenation catalyst is characterized as exhibiting at least a 5% reduction in the impurity relative to the fouled hydrogenation catalyst after contacting the flushing medium to the fouled hydrogenation catalyst for at least 1 hour, or at least 6 hours, or at least 12 hours, or at least 24 hours, or at least 2 days, or at least one week at the regeneration temperature and the regeneration pressure.

Clause 7. The method of clause 4, wherein the regenerated hydrogenation catalyst is characterized as exhibiting at least a 10% reduction, or at least a 15% reduction, or at least a 20% reduction, or at least a 25% reduction in the impurity relative to the fouled hydrogenation catalyst after contacting the flushing medium after contacting the flushing medium to the hydrogenation catalyst at the regeneration temperature and the regeneration pressure.

Clause 8. The method of any one of the preceding clauses, wherein the regeneration temperature is from 50° C. to 200° C.

Clause 9. The method of any one of the preceding clauses, wherein the regeneration pressure is from 20 psig to 300 psig.

Clause 10. The method of any one of the preceding clauses, wherein the flushing medium comprises a liquid phase and a vapor phase.

Clause 11. The method of clause 10, wherein the vapor phase comprises an oxygen content from 0.1% to 40% (v/v).

Clause 12. The method of clause 10, wherein the vapor phase comprises an oxygen content of at least 1% (v/v), or at least 5% (v/v), or at least 10% (v/v), or at least 15% (v/v), or at least 20% (v/v), or at least 25% (v/v).

Clause 13. The method of clause 10, wherein the inert gas is present in the vapor phase in an amount from 60% (v/v) to 99.5% (v/v).

Clause 14. The method of any one of the preceding clauses, wherein the inert gas is nitrogen.

Clause 15. The method of clause 10, wherein the vapor phase comprises air.

Clause 16. The method of any one of the preceding clauses, wherein the flushing medium comprises an oxygen to catalyst flux ratio (O₂/cat/hr) from 0.1*10⁻³ to 100*10⁻³ (mols/w/hr).

Clause 17. The method of clause 16 wherein the O₂/cat/hr is from 0.1*10⁻³ to 10*10⁻³ (mols/w/hr).

Clause 18. The method of any one of the preceding clauses, wherein the flushing medium comprises a water to catalyst flux ratio (H₂O/cat/hr) from 1 to 100 (w/w/hr).

Clause 19. The method of any one of the preceding clauses, wherein the flushing medium is free of hydrogen peroxide.

Clause 20. The method of any one of the preceding clauses, wherein the hydrogenation catalyst comprises a support and an active metal.

Clause 21. The method of any one of the preceding clauses, wherein the hydrogenation catalyst has one or more of the following properties:
(i) a total surface area of at least 500 m²/g;
(ii) a micropore surface area of at least 400 m²/g; and
(iii) a mesopore surface area of at least 30 m²/g.

Clause 22. The method of clause 21, wherein the regenerated hydrogenation catalyst has one or more of the following properties:

(i) the regenerated hydrogenation catalyst is characterized by retaining at least 70% of the micropore surface area of the fouled hydrogenation catalyst after contacting the flushing medium for at least 1 hour at the regeneration temperature and the regeneration pressure; and

(ii) the regenerated hydrogenation catalyst is characterized by retaining at least 70% of the mesopore surface area of the fouled hydrogenation catalyst after contacting the flushing medium for at least 1 hour at the regeneration temperature and the regeneration pressure.

Clause 23. The method of any one of the preceding clauses, wherein the hydrogenation catalyst is ruthenium on carbon (Ru/C).

Clause 24. A method for hydrogenating a biomass stream, the method comprising:

catalytically reacting a feedstock stream comprising water and sugar with hydrogen in the presence of a hydrogenation catalyst for a hydrogenation duration to produce a fouled hydrogenation catalyst;

replacing the feedstock stream with a flushing medium comprising water, oxygen, and an inert gas;

maintaining contact between the fouled hydrogenation catalyst and the flushing medium at a regeneration temperature and a regeneration pressure for a regeneration duration to produce a regenerated hydrogenation catalyst,

wherein the regenerated hydrogenation catalyst is characterized as retaining at least 70% of the total surface area of the fouled hydrogenation catalyst.

Clause 25. The method of clause 24, wherein the regenerated hydrogenation catalyst is characterized as retaining at least 70% of the total surface area of the fouled hydrogenation catalyst after contacting the flushing medium for at least 1 hour, or at least 6 hours, or at least 12 hours, or at least 24 hours, or at least 2 days, or at least one week at the regeneration temperature and the regeneration pressure.

Clause 26. The method of clause 24 or 25, wherein the regenerated hydrogenation catalyst is characterized as retaining at least 80%, or at least 90%, or at least 95% of the total surface area of the fouled hydrogenation catalyst after contacting the flushing medium to the hydrogenation catalyst for at least 1 hour at the regeneration temperature and the regeneration pressure.

Clause 27. The method of any one of the preceding clauses, wherein the regenerated hydrogenation catalyst is characterized as exhibiting at least at least a 5% reduction in an impurity relative to the fouled hydrogenation catalyst.

Clause 28. The method of clause 27, wherein the impurity is a sulfur-containing impurity.

Clause 29. The method of clause 27, wherein the regenerated hydrogenation catalyst is characterized as exhibiting at least a 5% reduction in the impurity relative to the fouled hydrogenation catalyst after contacting the flushing medium to the fouled hydrogenation catalyst for at least 1 hour, or at least 6 hours, or at least 12 hours, or at least 24 hours, or at least 2 days, or at least one week at the regeneration temperature and the regeneration pressure.

Clause 30. The method of clause 27, wherein the regenerated hydrogenation catalyst is characterized as exhibiting at least a 10% reduction, or at least a 15% reduction, or at least a 20% reduction, or at least a 25% reduction in the impurity relative to the fouled hydrogenation catalyst after contacting the flushing medium after contacting the flushing medium to the hydrogenation catalyst at the regeneration temperature and the regeneration pressure.

Clause 31. The method of clause 24, wherein the regenerated hydrogenation catalyst is characterized as retaining at least 70% of the conversion of the fouled hydrogenation catalyst for the sugar in the feedstock after contacting the flushing medium to the hydrogenation catalyst for at least 1 hour at the regeneration temperature and the regeneration pressure.

Clause 32. The method of clause 24, wherein the regenerated hydrogenation catalyst is characterized as retaining at least 80%, or at least 90%, or at least 95% of the conversion of the fouled hydrogenation catalyst for the sugar in the feedstock after contacting the flushing medium to the hydrogenation catalyst for at least 1 hour at the regeneration temperature and the regeneration pressure.

Clause 33. The method of any one of the preceding clauses, wherein the regeneration temperature is from 50° C. to 200° C.

Clause 34. The method of any one of the preceding clauses, wherein the regeneration pressure is from 20 psig to 300 psig.

Clause 35. The method of any one of the preceding clauses, wherein the flushing medium comprises a liquid phase and a vapor phase.

Clause 36. The method of clause 35, wherein the vapor phase comprises an oxygen content from 0.1% to 60% (v/v).

Clause 37. The method of clause 35, wherein the vapor phase comprises an oxygen content of at least 1% (v/v), or at least 5% (v/v), or at least 10% (v/v), or at least 15% (v/v), or at least 20% (v/v), or at least 25% (v/v).

Clause 38. The method of clause 35, wherein the inert gas is present in the vapor phase in an amount from 60% (v/v) to 99.5% (v/v).

Clause 39. The method of clause 38, wherein the inert gas is nitrogen.

Clause 40. The method of clause 35, wherein the vapor phase comprises air.

Clause 41. The method of any one of the preceding clauses, wherein the flushing medium comprises an oxygen to catalyst flux ratio (O₂/cat/hr) from 0.1*10⁻³ to 100*10⁻³ (mols/w/hr).

Clause 42. The method of clause 41 wherein the O₂/cat/hr is from 0.1*10⁻³ to 10*10⁻³ (mols/w/hr).

Clause 43. The method of any one of the preceding clauses, wherein the flushing medium comprises a water to catalyst flux ratio (H₂O/cat/hr) from 1 to 100 (w/w/hr).

Clause 44. The method of any one of the preceding clauses, wherein the flushing medium is free of hydrogen peroxide.

Clause 45. The method of any one of the preceding clauses, wherein the hydrogenation catalyst comprises a support and an active metal.

Clause 46. The method of any one of the preceding clauses, wherein the hydrogenation catalyst has one or more of the following properties:
(i) a total surface area of at least 500 m²/g;
(ii) a micropore surface area of at least 400 m²/g; and
(iii) a mesopore surface area of at least 30 m²/g.

Clause 47. The method of clause 46, wherein the regenerated hydrogenation catalyst has one or more of the following properties:

(i) the regenerated hydrogenation catalyst is characterized as retaining at least 70% of the hydrogenation catalyst's micropore surface area after contacting the flushing medium for at least 1 hour at the regeneration temperature and the regeneration pressure; and

(ii) the regenerated hydrogenation catalyst is characterized as retaining at least 70% of the hydrogenation catalyst's mesopore volume after contacting the flushing medium for at least 1 hour at the regeneration temperature and the regeneration pressure.

Clause 48. The method of any one of the preceding clauses, wherein the hydrogenation catalyst comprises ruthenium on carbon (Ru/C).

Clause 49. A method for hydrogenating a biomass stream, the method comprising:

catalytically reacting a feedstock stream comprising water and an oxygenated hydrocarbon (C₂₊O₁₊) with hydrogen in the presence of a hydrogenation catalyst for a hydrogenation duration to produce a fouled hydrogenation catalyst;

replacing the feedstock stream with a flushing medium comprising water and oxygen;

maintaining contact between the fouled hydrogenation catalyst and the flushing medium at a regeneration temperature and a regeneration pressure for a regeneration duration to produce a regenerated hydrogenation catalyst,

wherein the regenerated hydrogenation catalyst is characterized as retaining at least 70% of the conversion of the fouled hydrogenation catalyst for the oxygenated hydrocarbon in the feedstock after contacting the flushing medium to the hydrogenation catalyst for at least 1 hour at the regeneration temperature and the regeneration pressure.

Clause 50. The method of clause 49, wherein the oxygen is in the form of gaseous oxygen.

Clause 51. The method of clause 50, wherein the oxygen is in gaseous oxygen-containing gas stream.

Clause 52. The method of clause 51, wherein the oxygen-containing gas stream comprises air.

Clause 53. The method of any one of the preceding clauses, wherein the oxygenated hydrocarbon is a saccharide.

Clause 54. The method of any one of the preceding clauses, wherein the regeneration temperature is from 50° C. to 200° C.

Clause 55. The method of any one of the preceding clauses, wherein the regeneration pressure is from 20 psig to 300 psig.

Clause 56. The method of any one of the preceding clauses, wherein the flushing medium comprises an oxygen to catalyst flux ratio (O₂/cat/hr) from 0.1*10⁻³ to 100*10⁻³ (mols/w/hr).

Clause 57. The method of clause 56, wherein the O₂/car/hr is from 0.1*10⁻³ to 10*10⁻³ (mols/w/hr).

Clause 58. The method of any one of the preceding clauses, wherein the flushing medium comprises a water to catalyst flux ratio (H₂O/cat/hr) from 1 to 100 (w/w/hr).

Clause 59. The method of any one of the preceding clauses, wherein the flushing medium is free of hydrogen peroxide.

Clause 60. The method of any one of the preceding clauses, wherein the hydrogenation catalyst comprises a support and an active metal.

Clause 61. The method of clause 60, wherein the hydrogenation catalyst is ruthenium on carbon (Ru/C).

Clause 62. A method for producing a regenerated hydrogenation catalyst from a fouled hydrogenation catalyst, the fouled hydrogenation catalyst having at least one sulfur-containing impurity, the method comprising:

catalytically reacting a feedstock stream having at least one sulfur-containing impurity in the presence of a hydrogenation catalyst for a hydrogenation duration to produce the fouled hydrogenation catalyst,

replacing the feedstock stream with a flushing medium comprising water and oxygen,

maintaining contact between the fouled hydrogenation catalyst and the flushing medium at a regeneration temperature and a regeneration pressure for a regeneration duration to produce a regenerated hydrogenation catalyst,

wherein a concentration of the at least one sulfur-containing impurity in the regenerated hydrogenation catalyst is reduced relative to the fouled hydrogenation catalyst.

Clause 63. A method for producing a regenerated hydrogenation catalyst from a fouled hydrogenation catalyst, the fouled hydrogenation catalyst having at least one carbon-containing impurity, the method comprising:

catalytically reacting a feedstock stream having at least one carbon-containing impurity in the presence of a hydrogenation catalyst for a hydrogenation duration to produce the fouled hydrogenation catalyst,

replacing the feedstock stream with a flushing medium comprising water and oxygen,

maintaining contact between the fouled hydrogenation catalyst and the flushing medium at a regeneration temperature and a regeneration pressure for a regeneration duration to produce a regenerated hydrogenation catalyst,

wherein a concentration of the at least one carbon-containing impurity in the regenerated hydrogenation catalyst is reduced relative to the fouled hydrogenation catalyst.

Clause 64. The method of clause 62 or 63, wherein the regenerated hydrogenation catalyst is characterized as exhibiting at least a 5% reduction in the impurity relative to the fouled hydrogenation catalyst after contacting the flushing medium to the fouled hydrogenation catalyst for at least 1 hour, or at least 6 hours, or at least 12 hours, or at least 24 hours, or at least 2 days, or at least one week at the regeneration temperature and the regeneration pressure.

Clause 65. The method of clause 64, wherein the regenerated hydrogenation catalyst is characterized as exhibiting at least a 10% reduction, or at least a 15% reduction, or at least a 20% reduction, or at least a 25% reduction in the impurity relative to the fouled hydrogenation catalyst after contacting the flushing medium after contacting the flushing medium to the hydrogenation catalyst at the hydrogenation temperature and the hydrogenation pressure.

Clause 66. A method for producing a regenerated hydrogenation catalyst from a fouled hydrogenation catalyst, the fouled hydrogenation catalyst having at least one sulfur-containing impurity, the method comprising:

catalytically reacting a feedstock stream having at least one sulfur-containing impurity in the presence of a hydrogenation catalyst to produce the fouled hydrogenation catalyst,

replacing the feedstock stream with a flushing medium; and

maintaining contact between the fouled hydrogenation catalyst and the flushing medium at a regeneration temperature from 50° C. to 200° C., and a regeneration pressure from 20 psig to 300 psig for a regeneration duration to produce a regenerated hydrogenation catalyst,

wherein a concentration of the at least one sulfur-containing impurity in the regenerated hydrogenation catalyst is reduced relative to the fouled hydrogenation catalyst;

characterized in that the flushing medium comprises a liquid phase and a vapor phase, wherein the liquid phase comprises water and the vapor phase comprises oxygen.

1. A method for hydrogenating a biomass stream, the method comprising:
catalytically reacting a feedstock stream comprising water and an oxygenated hydrocarbon (C₂₊O₁₊) with hydrogen in the presence of a hydrogenation catalyst for a hydrogenation duration to produce a fouled hydrogenation catalyst;
replacing the feedstock stream with a flushing medium comprising water and oxygen;
maintaining contact between the fouled hydrogenation catalyst and the flushing medium at a regeneration temperature and a regeneration pressure for a regeneration duration to produce a regenerated hydrogenation catalyst,
wherein the regenerated hydrogenation catalyst is characterized as retaining at least 70% of the conversion of the hydrogenation catalyst for the oxygenated hydrocarbon in the feedstock after contacting the flushing medium to the hydrogenation catalyst for at least 1 hour at the regeneration temperature and the regeneration pressure.

2. The method of claim 1, wherein the oxygen is in the form of gaseous oxygen.

3. The method of claim 2, wherein the oxygen is in a gaseous oxygen-containing gas stream.

4. The method of claim 3, wherein the oxygen-containing gas stream comprises air.

5. The method of claim 1, wherein the oxygenated hydrocarbon is a saccharide.

6. The method of claim 1, wherein the regenerated hydrogenation catalyst is characterized as retaining more than 100% of the conversion of the fouled hydrogenation catalyst for the oxygenated hydrocarbon in the feedstock and retaining at least 70% of the conversion of the hydrogenation catalyst for the oxygenated hydrocarbon in the feedstock after contacting the flushing medium to the hydrogenation catalyst for at least 1 hour at the regeneration temperature and the regeneration pressure.

7. The method of claim 1, wherein the regeneration temperature is from 50° C. to 200° C.

8. The method of claim 1, wherein the regeneration pressure is from 20 psig to 300 psig.

9. The method of claim 1, wherein the flushing medium comprises an oxygen to catalyst flux ratio (O₂/cat/hr) from 0.1*10⁻³ to 100*10⁻³ (mols/w/hr).

10. The method of claim 9, wherein the O₂/car/hr is from 0.1*10⁻³ to 10*10⁻³ (mols/w/hr).

11. The method of claim 1, wherein the flushing medium comprises a water to catalyst flux ratio (H₂O/cat/hr) from 1 to 100 (w/w/hr).

12. The method of claim 1, wherein the flushing medium is free of hydrogen peroxide.

13. The method of claim 1, wherein the hydrogenation catalyst comprises a support and an active metal.

14. The method of claim 13, wherein the hydrogenation catalyst is ruthenium on carbon (Ru/C).

15. A method for producing a regenerated hydrogenation catalyst from a fouled hydrogenation catalyst, the fouled hydrogenation catalyst having at least one sulfur-containing impurity, the method comprising:
catalytically reacting a feedstock stream having at least one sulfur-containing impurity in the presence of a hydrogenation catalyst for a hydrogenation duration to produce the fouled hydrogenation catalyst,
replacing the feedstock stream with a flushing medium comprising water and oxygen,
maintaining contact between the fouled hydrogenation catalyst and the flushing medium at a regeneration temperature and a regeneration pressure for a regeneration duration to produce a regenerated hydrogenation catalyst,
wherein a concentration of the at least one sulfur-containing impurity in the regenerated hydrogenation catalyst is reduced relative to the fouled hydrogenation catalyst.

16. A method for producing a regenerated hydrogenation catalyst from a fouled hydrogenation catalyst, the fouled hydrogenation catalyst having at least one carbon-containing impurity, the method comprising:
catalytically reacting a feedstock stream having at least one carbon-containing impurity in the presence of a hydrogenation catalyst for a hydrogenation duration to produce the fouled hydrogenation catalyst,
replacing the feedstock stream with a flushing medium comprising water and oxygen,
maintaining contact between the fouled hydrogenation catalyst and the flushing medium at a regeneration temperature and a regeneration pressure for a regeneration duration to produce a regenerated hydrogenation catalyst,
wherein a concentration of the at least one carbon-containing impurity in the regenerated hydrogenation catalyst is reduced relative to the fouled hydrogenation catalyst.

17. The method of claim 15, wherein the regenerated hydrogenation catalyst is characterized as exhibiting at least a 5% reduction in the impurity relative to the fouled hydrogenation catalyst after contacting the flushing medium to the fouled hydrogenation catalyst for at least 1 hour, or at least 6 hours, or at least 12 hours, or at least 24 hours, or at least 2 days, or at least one week at the regeneration temperature and the regeneration pressure.

18. The method of claim 17, wherein the regenerated hydrogenation catalyst is characterized as exhibiting at least a 10% reduction, or at least a 15% reduction, or at least a 20% reduction, or at least a 25% reduction in the impurity relative to the fouled hydrogenation catalyst after contacting the flushing medium after contacting the flushing medium to the hydrogenation catalyst at the hydrogenation temperature and the hydrogenation pressure.

19. The method of claim 15, wherein the regeneration temperature is from 50° C. to 200° C.

20. The method of claim 15, wherein the regeneration pressure from 20 psig to 300 psig.

21-24. (canceled)
